(19) United States
(12) Patent Application Publication
(10) Pub. No.: US 2012/0302918 A1
(43) Pub. Date: Nov. 29, 2012

(54) ALL-IN-ONE DIABETES BLOOD GLUCOSE METER

(76) Inventor: William Dietz, Reston, VA (US)

(21) Appl. No.: 13/323,819

(22) Filed: Dec. 12, 2011

Related U.S. Application Data
(60) Provisional application No. 61/457,731, filed on May 23, 2011.

(51) Int. Cl. A61B 5/151

(52) U.S. Cl. 600/583

(57) ABSTRACT

Example embodiments relate to a portable blood glucose meter reader, including a housing member including an internal spring, a compartment configured to store test strips in alignment with the internal spring, a releasing member configured to dispense one of the plurality of stored test strips, and a plurality of storage members each configured to store a needle.

This application claims the benefit of U.S. provisional patent application No. 61/457,731, filed May 23, 2011, which is hereby incorporated by reference herein in its entirety.

### FIELD

Example embodiments relate to methods and systems regarding a blood glucose meter. More specifically, example embodiments relate to a system including elements or components for portable glucose testing.

### BACKGROUND

For a plurality of reasons, diabetes is increasing rapidly throughout the world. As the incidence of diabetes increases, a corresponding increase for diabetes monitoring and care will be necessary.

Many diabetics use glucose meters to check blood glucose levels. To test glucose levels in a conventional glucose meter, blood is placed on a disposable test strip and placed within the meter. The test strips are then coated with specific chemicals and combine with the glucose in the blood. The blood reader meter may then measure the amount of glucose present in the blood based on the reactions with the glucose and the specific chemicals.

However, conventional glucose meters do not include a blood glucose meter including all the components necessary to perform portable glucose testing a plurality of times.

### SUMMARY

Example embodiments relate to systems and methods utilizing a portable blood glucose meter reader. The portable blood glucose meter may include a housing member including an internal spring, a compartment configured to store test strips in alignment with the internal spring, a releasing member configured to dispense one of the plurality of stored test strips, and a plurality of storage members each configured to store a needle.

In a further example embodiment, the releasing member is a wheel configured to apply pressure to the one of the plurality of stored test strips if the wheel is rotated.

In another example embodiment, the releasing member is a slide bar configured to apply pressure to the one of the plurality of stored test strips if the slide bar is moved from a first position to a second position.

In a further example embodiment, one of the plurality of storage members is a clip disposed on the housing.

In another example embodiment, one of the plurality of storage members is an indentation disposed within the housing.

A further example embodiment may include a needle deployment mechanism within a blood glucose meter including a track with a stopper, a spring configured to compress and decompress to move the a needle along the track, and a needle housing with a concave outer surface configured to move along the track until the stopper inhibits the needle housing, such that only a tip of the needle protrudes from the needle housing.

### BRIEF DESCRIPTION OF THE DRAWINGS

The above and other features and advantages of example embodiments will become more apparent by describing in detail example embodiments with reference to the attached drawings. The accompanying drawings are intended to depict example embodiments and should not be interpreted to limit the intended scope of the claims. The accompanying drawings are not to be considered as drawn to scale unless explicitly noted.

FIG. 1 depicts a front view of a blood glucose meter;

FIG. 2 depicts another example embodiment of a blood glucose meter;

FIG. 3 depicts a back view of an example embodiment of a blood glucose meter;

FIG. 4 depicts a side view of the embodiment of a blood glucose meter as shown in FIG. 1 or 2;

FIG. 5 depicts a back view of another example embodiment of a blood glucose meter;

FIG. 6 depicts a bottom view of the example embodiment of a blood glucose meter as shown in FIG. 5;

FIG. 7 depicts an example embodiment of a needle deployment mechanism within a blood glucose meter;

FIG. 8 depicts a top view of the example embodiment of a needle deployment mechanism as shown in FIG. 7;

FIG. 9 depicts a front view of the example embodiment of a needle deployment mechanism as shown in FIGS. 7 and 8 within a blood glucose meter; and

FIG. 10 depicts a back view of the example embodiment of a needle deployment mechanism within a blood glucose meter as shown in FIG. 9.

### DETAILED DESCRIPTION OF THE DRAWINGS

Detailed example embodiments are disclosed herein. However, specific structural and functional details disclosed herein are merely representative for purposes of describing example embodiments. Example embodiments may, however, be embodied in many alternate forms and should not be construed as limited to only the embodiments set forth herein.

Accordingly, while example embodiments are capable of various modifications and alternative forms, embodiments thereof are shown by way of example in the drawings and will herein be described in detail. It should be understood, however, that there is no intent to limit example embodiments to the particular forms disclosed, but to the contrary, example embodiments are to cover all modifications, equivalents, and alternatives falling within the scope of example embodiments. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments. As used herein, the term “and/or” includes any and all combinations of one or more of the associated listed items.

It will be understood that when an element is referred to as being “connected” or “coupled” to another element, it may be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between”, “adjacent” versus “directly adjacent”, etc.).

The terminology used herein is for the purpose of describing particular embodiments only and is not intended to be limiting of example embodiments. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

As discussed in greater detail below, embodiments herein relate to a glucose blood meter including desired or required elements for portable glucose testing, such as equipment to puncture skin with a needle to produce bodily fluids or blood, a plurality of needles, a plurality of test strips, a strip dispenser, and a test strip reader to read blood test results.

In other words, embodiments herein may be related with an all-in-one glucose blood meter. Accordingly, the glucose blood meter in the below embodiments allow for convenient transport of the glucose blood meter that limits or prevents the loss of any component of the system that would prevent or limit the ability to check or monitor blood glucose levels. Thus, the below example embodiments reduce, limit or prevent the probability that a glucose blood meter is not accompanied with test strips or needles.

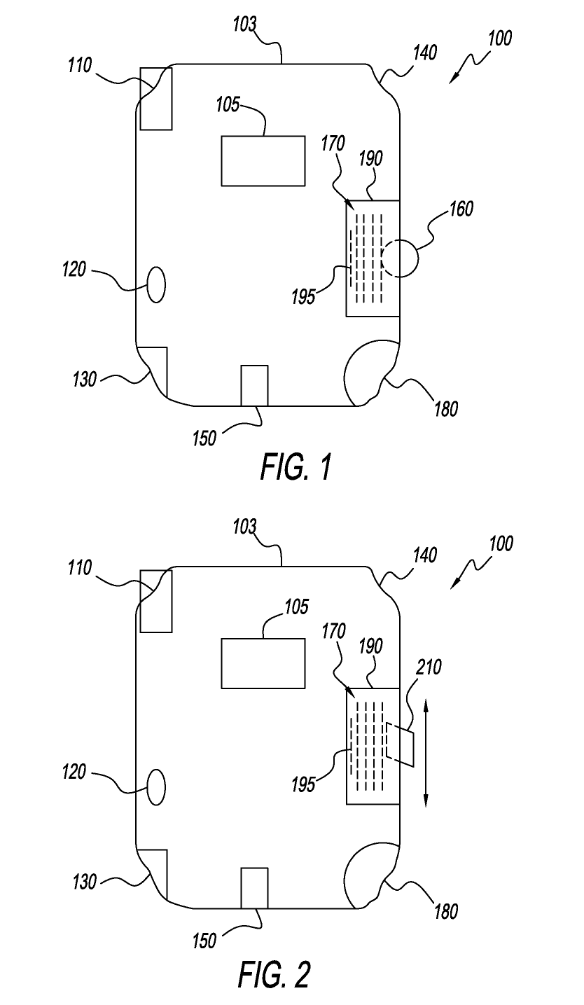

FIG. 1 represents a front view of apparatus 100. Apparatus 100 may be a portable diabetes blood reader meter allowing people to measure their blood-sugar levels a plurality of times at various locations. The apparatus 100 may include a housing 103 substantially comprised of plastic; however, other embodiments may be formed or comprised of any other known solid material.

Apparatus 100 may include a display 105, a primer component 110, a needle release button 120, a needle deployment component 130, a strip storage component 140, a wheel 160, strips 170 and a strip removal component 180. It should be appreciated that apparatus 100 may further be operatively connected to other devices such as computers, processors, speakers, and the like.

Display 105 may be configured to display the test results, as well as other information. Display 105 may be a LCD display or any other electronic display capable of displaying blood-sugar level test results, such as a LED display.

Primer 110 is a known component of existing glucose meters that readies the needle for deployment by the needle release button 120. To use the meter, primer 110 is activated, then the needle release button 120 is pressed, which projects the tip or head of the testing needle out of the deployment slot 130 for use (pricking a user's skin to obtain blood for testing). In further example embodiments, the primer may be a plastic pull tab.

Strips 140 may be stored within housing 103. The strips 140 may be placed in housing 103 in strip storage 170 in alignment with a loading spring 195. As more strips 140 are placed within the housing 103, the loading spring 195 may compress.

Furthermore, tension caused by compressing the loading spring 195 and by scrolling wheel 160 against the strips 140, may cause a strip 140 to be dispensed or unloaded out of slot 180. Also, wheel 160 may be substantially comprised of plastic or rubber. In another example embodiment, the wheel 160 may be flat, cross-stitched, or contain ridges to propel the strip out of slot 180 when the wheel 160 is scrolled.

In further example embodiments, housing 103 may include a releasable cover 190, which may be either on the front surface or back surface of housing 103. Upon releasing the releasable cover 190, a user may insert or withdraw the strips 140 in bulk or individually from strip storage 170. In other words, a user may load or withdraw any numbers of strips 140 from strip storage 170 if the releasable cover is removed or released from housing 103.

FIG. 2 depicts another example embodiment of apparatus 100. In FIG. 2, the wheel 160 of FIG. 1 may instead be a knob or a slide bar configured to move back and forth to expel or dispense strips 140 from the housing 103. In such an embodiment, the slide bar 210 at rest does not contact the test strips 140, or does so only lightly, so that it can slide freely. When pressure is exerted on the slide by the user, the slide bar 210 is pressed against the top strip such that movement of the slide bar 210 also moves the strip 140. In other example embodiments, wheel 160 may instead be any apparatus configured to dispense the strips 140 from the housing 103.

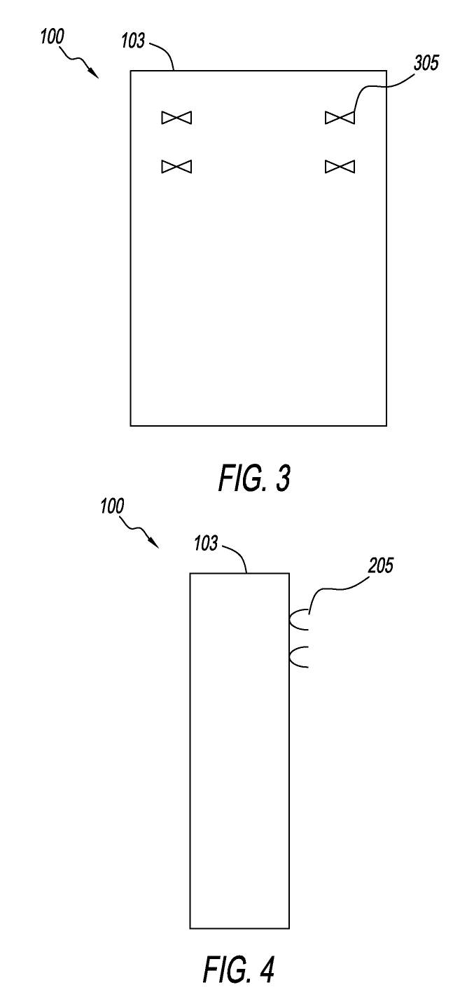

FIG. 3 depicts a back view of an example embodiment of apparatus 100. In FIG. 3, housing 103 includes clips 305 configured to hold extra needles.

Clips 305 may be any holding device disposed on housing 103 configured, formed or shaped to receive and hold or support needles or needle casings. The clips 305 may be positioned on a back side of housing 103, such that extra needles are stored in a horizontal and/or vertical orientation with respect to a base of the housing 103. Furthermore, a plurality of clips 305 may store, hold, or receive an individual needle or needle casing to provide extra support or stability. More so, the clips 305 may be various devices for gripping or holding needles, such as a fastener or clasp.

FIG. 4 depicts a side view of the embodiment of apparatus 100 as shown in FIG. 1 or 2. In FIG. 4, housing 103 includes clips 305 configured to hold or store needles or needle casings in a horizontal orientation with respect to a base of the housing 103. Furthermore, as shown in FIG. 4 the clips 305 may be disposed on housing 103.

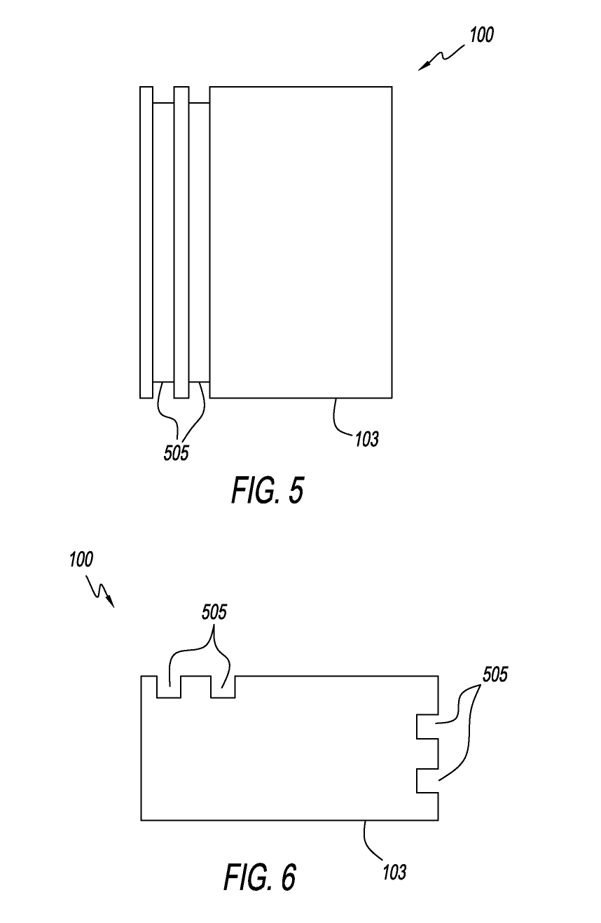

FIG. 5 depicts a back view of another example embodiment of apparatus 100. In FIG. 5, housing 103 includes indentations or inverted portions 505 configured, formed or shaped to receive and hold needles or needle casings. More specifically, the indentations 505 may be approximately a width of a needle, or a width of a case containing a needle, and may be approximately the length of a needle. In other words, the indentations 505 may be disposed within housing 103 to hold extra needles.

FIG. 6 depicts a bottom view of the example embodiment of apparatus as shown in FIG. 5. More specifically, FIG. 6 depicts indentations 505 configured or placed on both the back and side surfaces of housing 103. In further example embodiments, the indentations 505 may be placed on either or both sides of the housing 103, as well as on a back surface of the housing 103.

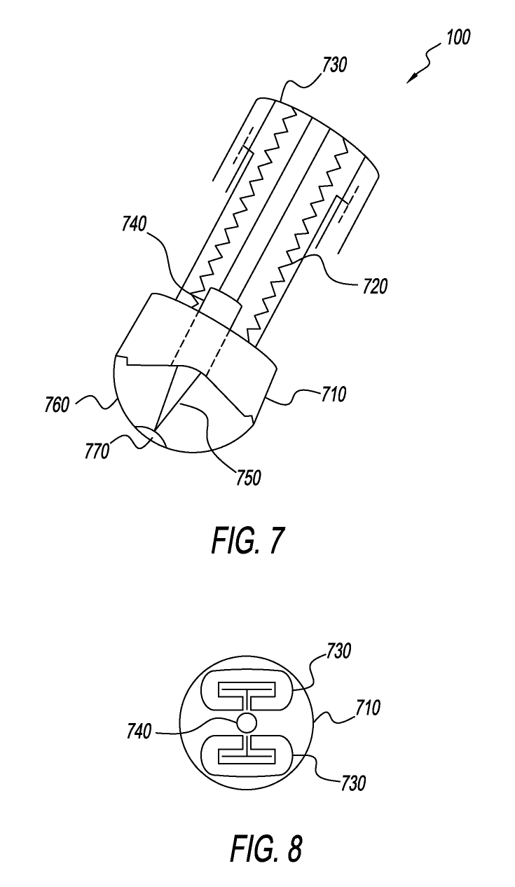

FIG. 7 depicts an example embodiment of a needle deployment mechanism 700 that may be located within housing 103 of FIG. 1 or 2.

Needle deployment mechanism 700 may include needle housing 710, a spring 720, a track 730, a needle holder 740, a needle 750 and a cap 760.

Needle holder 740 may hold needle 750 in a fixed position while needle housing 710 and cap 760 are configured to slide along track 730.

Track 730 may be aligned with an edge of housing 103. More so, the track 730 may be configured such that the needle housing 710 and cap 760 may move along the track 730. Spring 720 may be substantially aligned with the track 730 and connected to needle housing 710, and may be compressed or extended as needle housing 710 moves along the track 730. More specifically, if the spring 720 is compressed the needle housing 710 may be in a top position along the track 730, whereas when the spring 720 is decompressed the needle holder 710 may be in a bottom position along the track 730.

Needle housing 710 may be configured to move along track 730, and may include a surface facing away from housing 103 that is concave or curved inward. Track 730 may include a stopper configured to stop the needle housing 710 and cap 760 when the spring 720 is compressed. Accordingly, the stopper inhibits needle housing 710 from moving further inward along the track 730. Therefore, when the needle housing 710 is adjacent to the stopper only the tip or head of the needle 750 is protruding from the needle housing 710 and cap 760 through hole 770. In other words, needle housing 710 may slide or move along the track 730 to the stopper to determine a length of the needle 750 that may prick the skin of the user.

The widest portion of the concave surface of needle housing 710 may be substantially aligned with an edge of housing 103 when needle housing 710 is adjacent to the stopper. Because the outer surface of needle housing 710 is concave a user may roll housing 103 along the users skin so that only the tip of the needle 750 pricks the skin of the user.

In further example embodiments, cap 760 may cover the portion of the needle 750 protruding from needle housing 710 when the needle 750 is not in use. The cap 760 may be removed in order to replace needle 750.

FIG. 8 depicts a top view of the example embodiment of a needle deployment mechanism as shown in FIG. 7. The cross-section of the track 730 can be clearly seen.

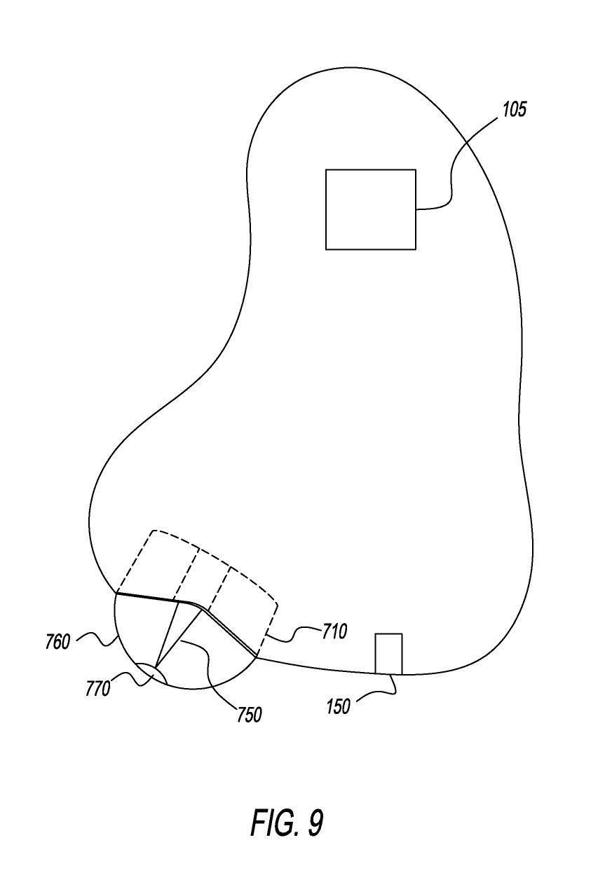

FIG. 9 depicts a front view of the example embodiment of a needle deployment mechanism as shown in FIGS. 7 and 8 within a blood glucose meter. This meter has an ergonomic design and is shaped with a rounded end for use of the needle. The rounded needle end can be rolled along a user's arm and as the needle housing 710 contacts the arm, it will slide back until it hits its stop, leaving the needle 750 exposed so that it pricks the arm as the arm rolls across the end of the meter. Thus, a user does not need to worry about the proper force with which to apply the needle.

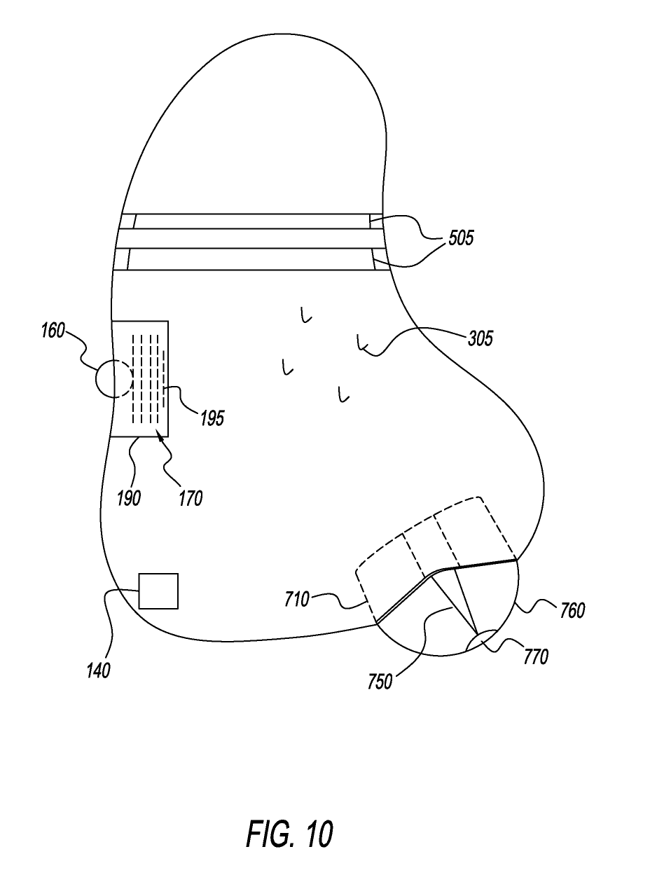

FIG. 10 depicts a back view of this example embodiment, showing the inclusion of needle clips 305 and indentations 505 and strip dispenser mechanisms 140, 160, 170, 190, 195. Of course all the features discussed above with regard to the embodiment of FIG. 1 can be applied to this embodiment as well, as for example a sliding bar 210 can be substituted for wheel 160, clips 305 and indentations 505 may be on the meter separately or in combination, etc.

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments. While the embodiments may be illustrated by using a particular embodiment, this is not and does not limit the invention to any particular embodiment and a person of ordinary skill in the art will recognize that additional embodiments are readily understandable and are a part of the example embodiments.

It will also be appreciated that one or more of the elements depicted in the drawings/figures can also be implemented in a more separated or integrated manner, or even removed or rendered as inoperable in certain cases, as is useful in accordance with a particular application. Additionally, any signal arrows in the drawings/figures should be considered only as exemplary, and not limiting, unless otherwise specifically noted.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component.

What is claimed:

1. A portable blood glucose meter, comprising:
a housing member including an internal spring;
a compartment configured to store test strips in alignment with the internal spring;
a releasing member configured to dispense one of the plurality of stored test strips; and
a plurality of storage members each configured to store a needle.

2. The portable blood glucose meter reader of claim 1, wherein the releasing member is a wheel configured to apply pressure to the one of the plurality of stored test strips if the wheel is rotated.

3. The portable blood glucose meter reader of claim 1, wherein the releasing member is a slide bar configured to apply pressure to the one of the plurality of stored test strips if the slide bar is moved from a first position to a second position.

4. The portable blood glucose meter reader of claim 1, wherein one of the plurality of storage members is a clip disposed on the housing.

5. The portable blood glucose meter reader of claim 1, wherein one of the plurality of storage members is an indentation disposed within the housing.

6. The portable blood glucose meter of claim 1, further comprising a needle deployment mechanism within the blood glucose meter comprising a track with a stopper, a needle housing with a concave outer surface configured to move along the track until the stopper inhibits the needle housing, such that only a tip of the needle protrudes from the needle housing, and a spring configured to compress and decompress to move the needle housing along the track.

7. The portable blood glucose meter of claim 6, wherein a side of the blood glucose meter from which the needle deployment mechanism projects is rounded for rolling along a user's skin.
